(19) United States
(12) Patent Application Publication
(10) Pub. No.: US 2008/0047549 A1
(43) Pub. Date: Feb. 28, 2008

(54) SURGICAL EQUIPMENT SUPPORTING FRAMES AND ATTACHMENTS FOR SAME

(76) Inventors: Michael Edward Theran, Bethany, CT (US); John Francis Irving, Hamden, CT (US)

Correspondence Address: F. CHAU & ASSOCIATES, LLC, 130 WOODBURY ROAD, WOODBURY, NY, 11797, US

(21) Appl. No.: 11/831,536

(22) Filed: Jul. 31, 2007

Related U.S. Application Data
(60) Provisional application No. 60/834,967, filed on Aug. 2, 2006.

(51) Int. Cl. A61F 5/28

(52) U.S. Cl. 128/99.1

(57) ABSTRACT

A surgical utility tube, comprising a plurality of members, wherein the plurality of members are joined together to form a continuous first tube, wherein the first tube is bendable to a plurality of angles along a length of the first tube, and wherein the first tube locks at each of the plurality of angles, and a second tube positioned in the first tube.

### CROSS-REFERENCE TO RELATED APPLICATION

This application claims the benefit of Provisional Application Ser. No. 60//834,967, filed on Aug. 1, 2006, the contents of which are herein incorporated by reference in their entirety.

### BACKGROUND OF THE INVENTION

1. Technical Field

The present disclosure relates to surgical equipment supporting frames and attachments for same.

2. Discussion of the Related Art

Devices to support surgical instruments, which attach to, for example, an operating table, are known. These devices are positioned on or near an operating table to allow an operating physician or assistant access to surgical tools and utilities, such as, for example, suction tubes, irrigation tubes, scopes, smoke exhaust ducts, cautery, pens, common hand tools and auxiliary lights. Such devices also enable the operator or assistant to use the surgical tools and utilities when needed, and retain them in a position of access when not in use.

However, the known devices have limited ranges of motion, may not be suitable for more than one type of surgery, and may not be equipped to hold multiple surgical tools or utilities. For example, a known device may be designed for a specific type of surgical procedure, such as laparoscopic surgery, and is not useful for other surgeries. In addition, a known device mounted to an operating table can have a limited range of motion, and while useful for a surgery at one part of the body, for example, the head, is not useful for a surgery at another part body, such as the legs, which are outside of the device's range of motion.

Known devices are also designed for specific purposes, such as retraction, and are limited to such functions. Such devices are not equipped to handle multiple functions that may be needed for different surgeries.

Accordingly, there is need for a device to support surgical tools, which has an increased range of motion, is universal to multiple types of surgical procedures, and is capable of supporting a variety of surgical tools for multiple procedures.

### SUMMARY OF THE INVENTION

Embodiments of the present invention provide medical practitioners, such as surgeons, with facilitated access to tools and other utilities to improve operating room efficiency by reducing labor requirements or by reducing the time to perform various procedures. Tools and utilities such as suction tubes, irrigation tubes, scopes, smoke exhaust ducts, cautery pens, common hand tools and auxiliary lights are examples of the types of tools and utilities that can be supported.

A surgical utility tube, in accordance with an embodiment of the present invention, comprises an outer surface, an inner surface, a wall formed between the inner and outer surfaces, and a plurality of wires formed in the wall between the outer and inner surfaces.

The wires may be spaced apart at predetermined intervals around the wall of the tube. The tube may be used for at least one of suction and irrigation. The tube may be used as a smoke duct or to support a light.

The wires at a first end of the tube may have less rigidity than the wires at a second end of the tube. The wires at an end of the tube may have less rigidity than the wires at a portion of the tube between ends of the tube.

The first end of the tube may be attached to a frame positioned at a side of an operating table and a second free end of the tube may be self-suspended over the operating table.

The surgical utility tube may further comprise a pig tail wire attached to the tube, and a surgical tool attached to the pig tail wire.

The surgical utility tube may further comprise a recoiling spool attached to the tube, and a surgical tool attached to the recoiling spool.

A surgical utility tube, in accordance with an embodiment of the present invention, comprises a plurality of members, wherein the plurality of members are joined together to form a continuous first tube, wherein the first tube is bendable to a plurality of angles along a length of the first tube, and wherein the first tube locks at each of the plurality of angles, and a second tube positioned in the first tube.

The second tube may be flexible and may be used for at least one of suction and irrigation, or as a smoke duct.

The plurality of members may comprise a plurality of interlocking knuckles or adjacent sections of a helix.

The first end of the first tube may be attached to a frame positioned it a side of an operating table and a second free end of the first tube may be self-suspended over the operating table.

The surgical utility tube may further comprise a pig tail wire attached to the first tube, and a surgical tool attached to the pig tail wire.

The surgical utility tube may further comprise a recoiling spool attached to the first tube, and a surgical tool attached to the recoiling spool.

### BRIEF DESCRIPTION OF THE DRAWINGS

Exemplary embodiments of the present invention will be described below in more detail, with reference to the accompanying drawings, of which:

FIG. 1 is a perspective view of a surgical equipment supporting frame in an extended position, according to an embodiment of the present invention;

FIG. 2 is a perspective view of a surgical equipment supporting frame in a partially extended position, according to an embodiment of the present invention;

FIG. 3 is a perspective view of a surgical equipment supporting frame in a retracted position, according to an embodiment of the present invention;

FIG. 4 is a perspective view of a surgical equipment supporting frame in a partially extended position and with a patient on the operating table, according to an embodiment of the present invention;

FIG. 5 is a perspective view of a surgical equipment supporting frame in a retracted position and with a patient on the operating table, according to an embodiment of the present invention;

FIG. 6 is an overhead perspective view of a surgical equipment supporting frame in an extended position, according to an embodiment of the present invention;

FIGS. 7-10 are perspective views of an interface for consumable tools, according to an embodiment of the present invention;

FIG. 11 is a perspective view of a surgical equipment supporting frame in an extended position, according to an embodiment of the present invention;

FIG. 12 is a perspective view of a surgical equipment supporting frame, according to an embodiment of the present invention;

FIG. 13 is a perspective view of a surgical equipment supporting frame, according to embodiment of the present invention;

FIG. 14 is a perspective view of a surgical equipment supporting frame, according to an embodiment of the present invention;

FIG. 15 is a perspective view of a surgical equipment supporting frame, according to an embodiment of the present invention;

FIG. 16 is a cross-sectional view of a co-extruded tube, according to an embodiment of the present invention;

FIG. 17 shows a helix tube in snapped and unsnapped configurations, according to an embodiment of the present invention;

FIG. 18 shows a tube including interlocking knuckles, according to an embodiment of the present invention;

FIG. 19 shows a tube including interlocking knuckles and a co-extruded tube at an end thereof, according to an embodiment of the present invention;

FIG. 20 shows a tube including interlocking knuckles including a standard tube therein, according to an embodiment of the present invention;

FIG. 21 shows a surgical tool attached to a pig tail wire, according to an embodiment of the present invention; and

FIG. 22 shows a surgical toot attached to a recoiling spool, according to an embodiment of the present invention.

### DETAILED DESCRIPTION OF EXEMPLARY EMBODIMENTS

Exemplary embodiments of the present invention now will be described more fully hereinafter with reference to the accompanying drawings. This invention may, however, be embodied in many different forms and should not be construed as limited to the embodiments set forth herein.

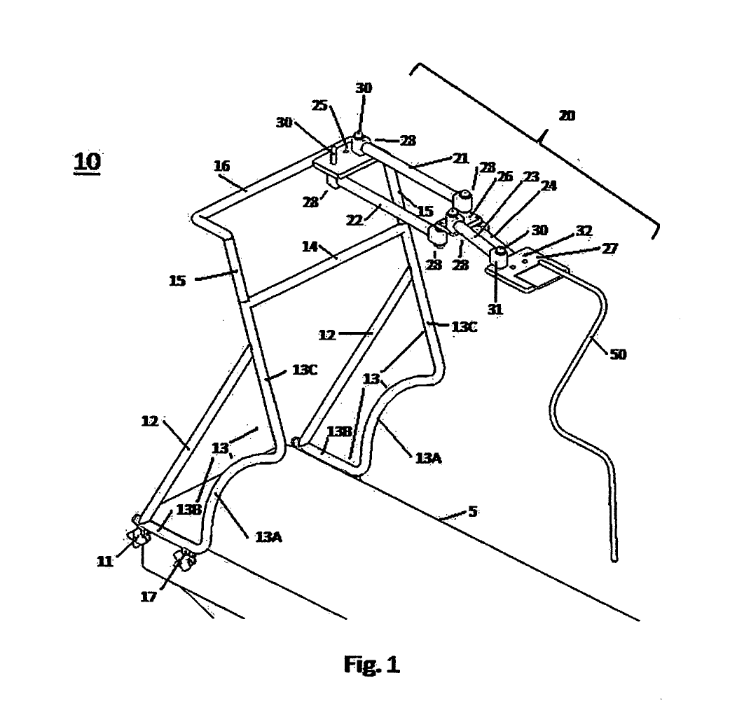

Referring to FIGS. 1-6, a surgical equipment supporting frame 10 is shown attached to an operating table 5. The frame 10 is attached to the operating table 5 using fixing devices, such as, for example, screws, bolts, and the like. For example, as shown in FIGS. 1-5, screws 11 having a gripping handle, which can be hand tightened, are used. The screws 11 allow for easy installation and removal of the frame 10 from the operating table 5. The frame 10 includes a plurality of branches forming the structure of the frame. For example, the frame includes branches 12, 13, 14, 15 and 16. The branches 12-16 can be molded as a single piece, and/or formed in separate parts and subsequently joined by, for example, welding, press fitting, and/or snap fitting, using, for example, spring-biased balls snapped into recesses.

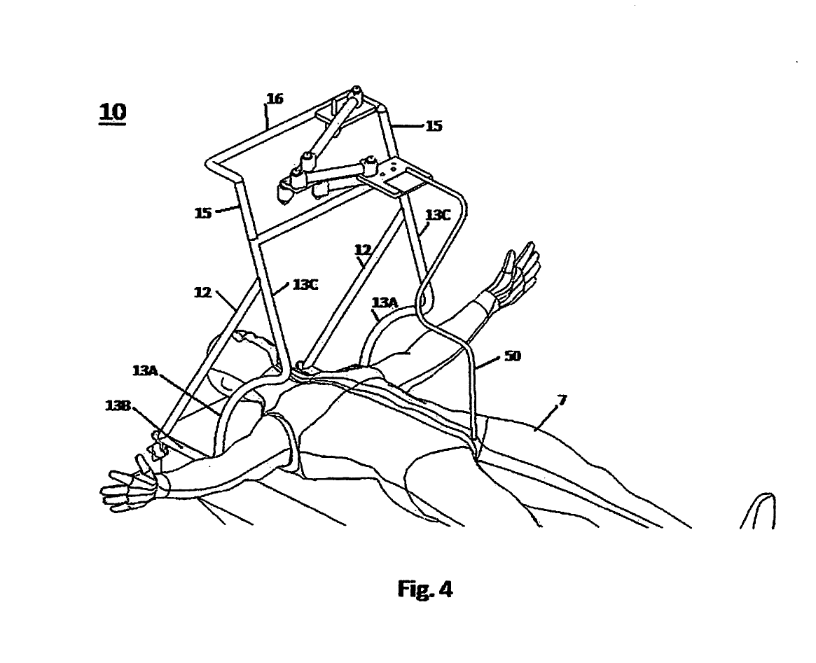

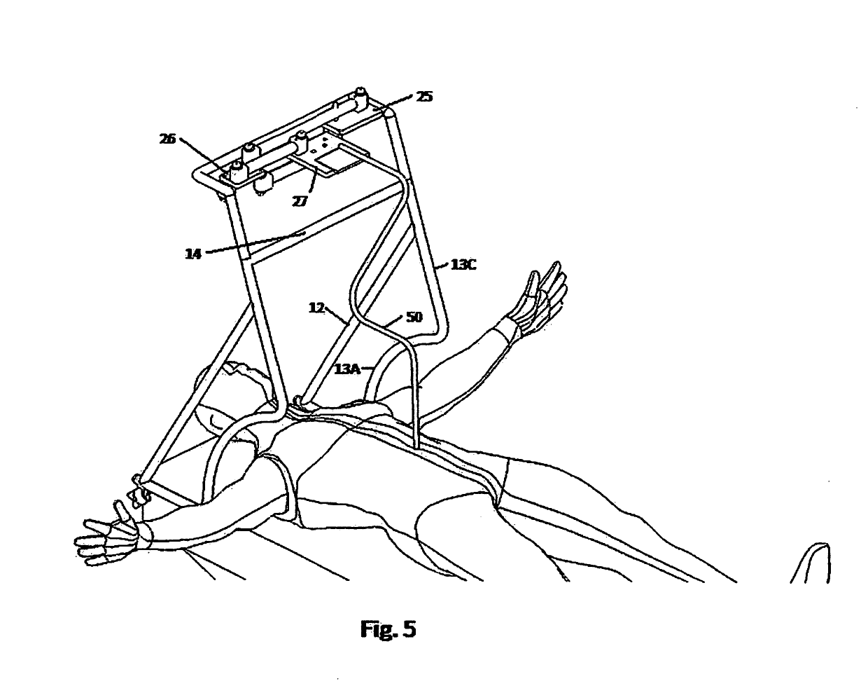

Referring to FIGS. 4 and 5, the branches 13 each include a curved portion 13A, which is curved around the shoulders and/or arms of a patient 7, so as not to obstruct access to these parts of the body. Branches 13 also include a lower portion 13B substantially parallel to a side of the operating table 5 and an upper portion 13C oblique to the operating table 5. The curved portion 13A is positioned between the upper and lower portions 13C and 13B. The branch 12 extends between the upper and lower portions 13C and 13B behind the curved portion 13A. The fixing devices 11 are attached to the frame via, for example, holes in the lower portion 13B or loops 17 extending from the lower portion 13B.

Branch 14 extends substantially perpendicular to and between branches 13C. Branches 15 extend in-line from respective branches 13C, and U-shaped branch 16 attaches to branches 15 and oblique to branches 15. The branches 14 and 16 are sufficiently high above the patient 7 so as to provide an operator with required clearance for unobstructed access to the body. Some of the branches may be removed depending on the use of the frame 10. For example, if the operation is on the head portion of the body, branch 12 may be removed to provide the operator with increased access to the head. In addition, if branch 16 must be positioned closer to the patient 7, branches 15 could be removed to reduce the height of branch 16.

As shown in FIGS. 1-6, the frame 10 includes an arm portion 20. The arm portion 20 includes a plurality of limbs 21, 22, 23, 24 connected to base portions 25, 26 and 27. For example, base portion 25 is connected to branch 16 by, for example, molding, welding, adhesive, or a mechanical fixing device, and limbs 21 and 22 extend from base portion 25. The limbs 21 and 22 extend from upper and lower sides of the base portion 25 respectively. The limbs 21 and 22 are connected to the base portion 25 at rotatable joints 28. The limbs 21 and 22 end at base portion 26, and are connected to base portion 26 at respective upper and lower sides of the base portion 26 using the rotatable joints 28. Similarly, limbs 23 and 24 extend from upper and lower sides of base portion 26 to upper and lower sides of base portion 27, respectively. The limbs 23 and 24 are connected to the base portions 26 and 27 using the rotatable joints 28.

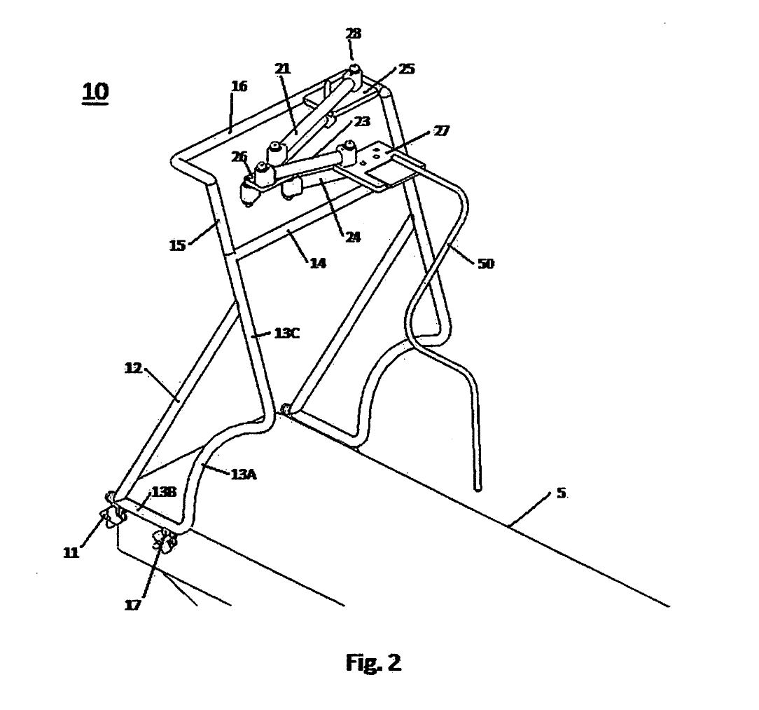

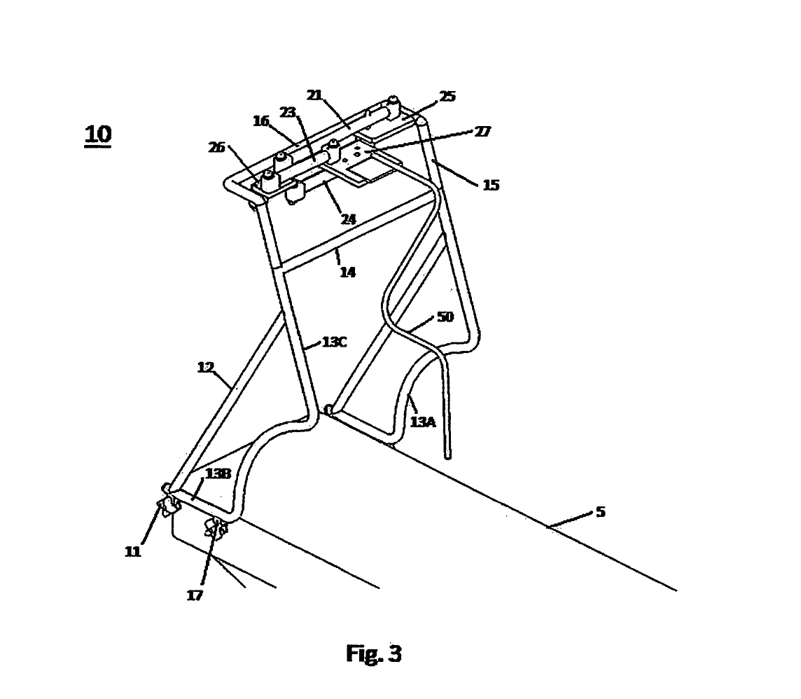

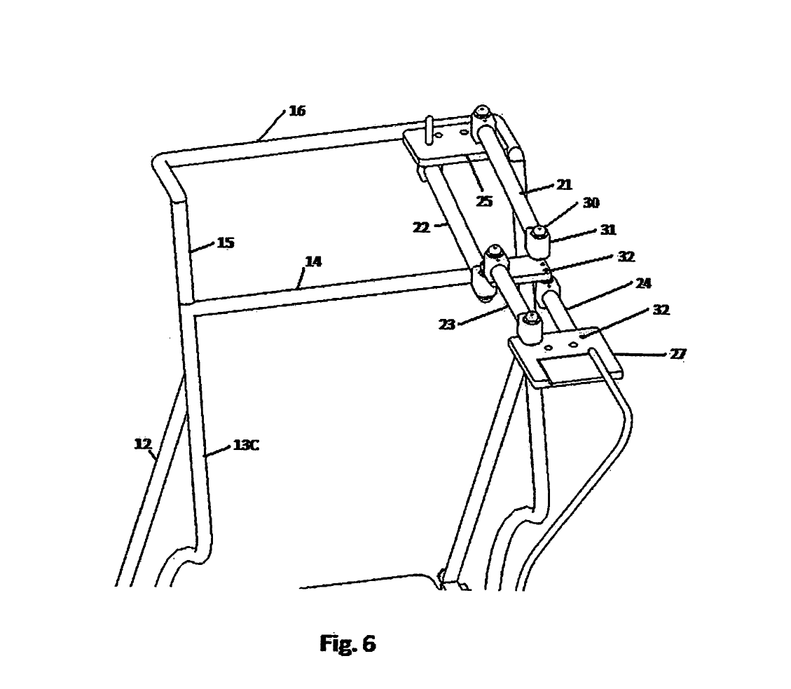

The arm portion 20, like a human arm attached to a shoulder (e.g., base portion 25) and having an elbow (e.g., base portion 26) and a wrist (e.g., base portion 27), can be fully extended, as shown in FIGS. 1 and 6, partially extended, as shown in FIGS. 2 and 4, and folded, as shown in FIGS. 3 and 5. The arm portion 20 can be extended and fixed at a plurality of positions between the non-extended and fully extended positions. As shown in FIGS. 4 and 5, the plurality of positions enable an operator to reach the surgical tools to different parts of the body of the patient 7. For example, referring to FIGS. 4 and 5, when in a partially extended position, the surgical tool 50 can reach portions of the body closer to the feet than when in the folded position.

The rotatable joints 28 include a fixing device 30, such as a screw or a bolt, penetrating a sleeve 31 into which the end of a limb 21-24 is inserted and fixed. The sleeve 31 may form part of the limb 21-24. Alternatively, the sleeve 31 can be omitted and the limb 21-24 can include a hole through which the fixing device 30 passes. The base portions 25-27 include holes 32 for receiving the fixing devices 30. The arrangement of the fixing device 30 permits side to side movement of the limbs 21-24 about the joints 28. A limb may rotate approximately 180 degrees to form an arc, wherein the rotatable joint 28 forms the center of the arc, and the length of the limb is the radius of the arc. For example, the limb 21 may form an arc from a right side of the operating table 5 to a left side of the operating table 5 across the width of the operating table 5.

According to an embodiment of the present invention, upon locating the arm portion 20 in a desired position, the fixing devices 30 may be adjustable to be tightened and lock the arm portion 20 in the desired position. Alternatively, the arm portion 20 and the fixing devices 30 may be readily moved from side to side to change the location of the arm portion 20. According to an embodiment, the rotatable joints 28 may rotate around multiple axes, so as to permit, for example, side to side and up and down movement. To permit rotation around multiple axes, a ball and socket joint may be used.

The base portion 27 includes an interface 40 at the end of the base portion 27 opposite the end where the limbs 23 and 24 are attached. The interface 40 is used to mount surgical tools 50, such as, for example, suction tubes, irrigation tubes, scopes, smoke exhaust ducts, cautery pens, common hand tools and auxiliary lights, on the frame 10. Depending on the type of surgery, a kit of one or more tools 50 can be attached to the frame 10 via the interface 40. For example, a kit may include a plurality of consumable tubes used for suction, smoke exhaust, and irrigation, and be designated according to the type of surgery, for example, as an orthopedic kit or a cardiac kit. Further, each kit can be discarded after the surgery is completed. According to an embodiment, each kit includes a sterile sheath (not shown) running therefrom, which covers the arm portion 20 after the kit and tools 50 are mounted to the frame 10 via the interface 40. For example, the sterile sheath may be attached to the mating cover portion 43, and extended therefrom to cover the arm portion 20.

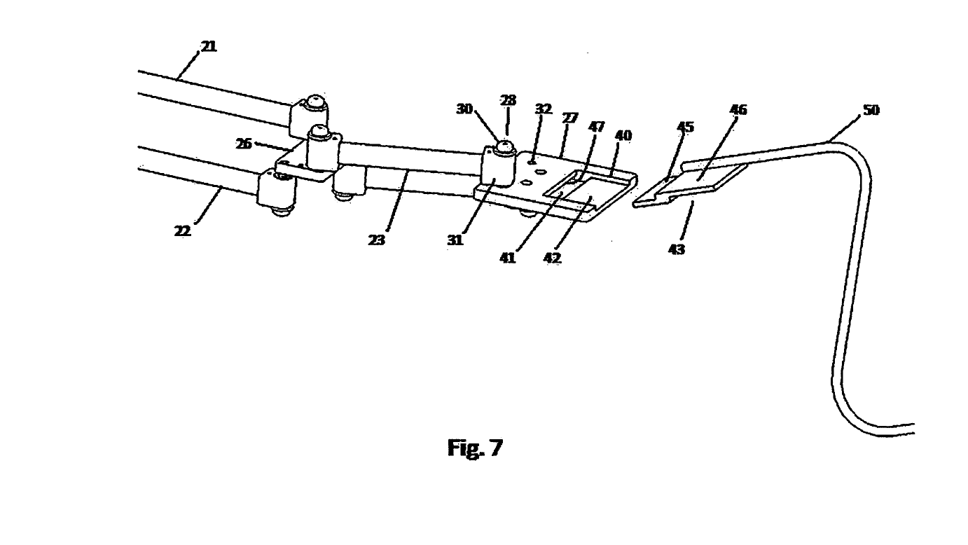

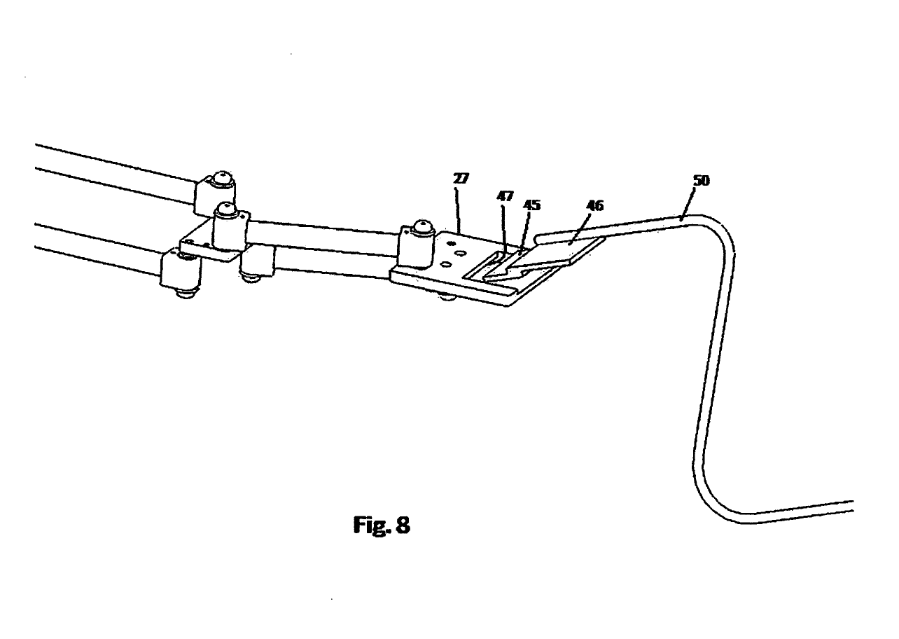

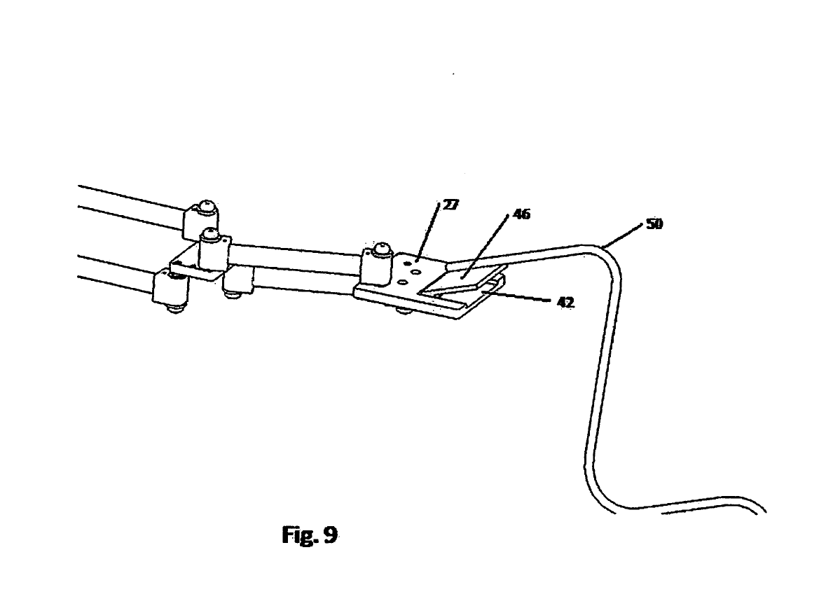

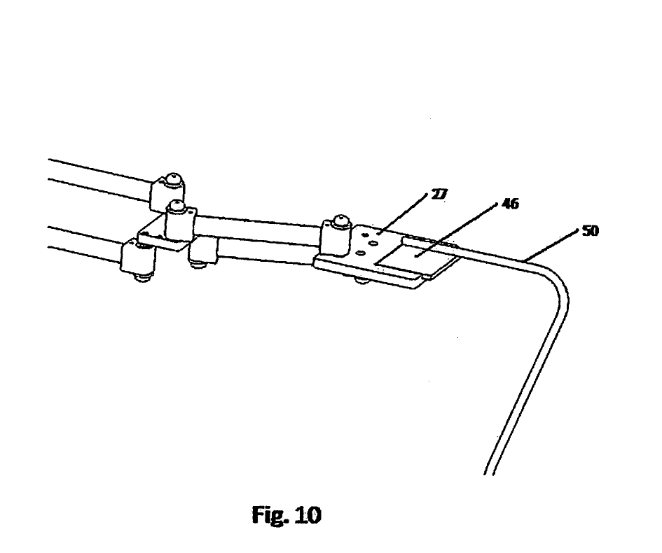

Referring to FIGS. 7-10, the interface 40 includes a floor having portions 41 and 42 at different heights, with a step between portions 41 and 42. The interface 40 receives a cover portion 43, which sits on the stepped floor and functions as a mating interface portion. The cover portion 43 includes portions 45 and 46, which correspond to portions 41 and 42, respectively. Like the portions 41 and 42, the portions 45 and 46 are at different heights, and include a step between them. Portion 46 overlaps portion 45, and part of portion 45 extends through an opening at the back 47 of the interface 40 to lock the cover portion 43 in place. Alternatively, other types of interfaces are contemplated, including, for example, snap-in or press-fit tabs, adhesives, latches, button or switch operated quick release mechanisms, and VELCRO type fasteners.

The cover portion 43 may have one or more surgical tools 50 attached thereto. Further, multiple interfaces 40 may be positioned on base portion 27 to mate with corresponding cover portions 43 including one or more surgical tools 50 attached thereto. The respective sizes of the interface and cover portions 40, 43 may be reduced to accommodate multiple interfaces on the base portion 27.

Tubes or wires providing conduits for or power to the surgical tools can be attached to the surgical tools 50 at, for example, portion 46. The tubes or wires can be extended to be dressed along and adjacent any or all of the limbs 21-24 of the arm portion 20, and any or all of the branches 12-16 of the frame 10 to their respective deposit tanks, and/or power sources.

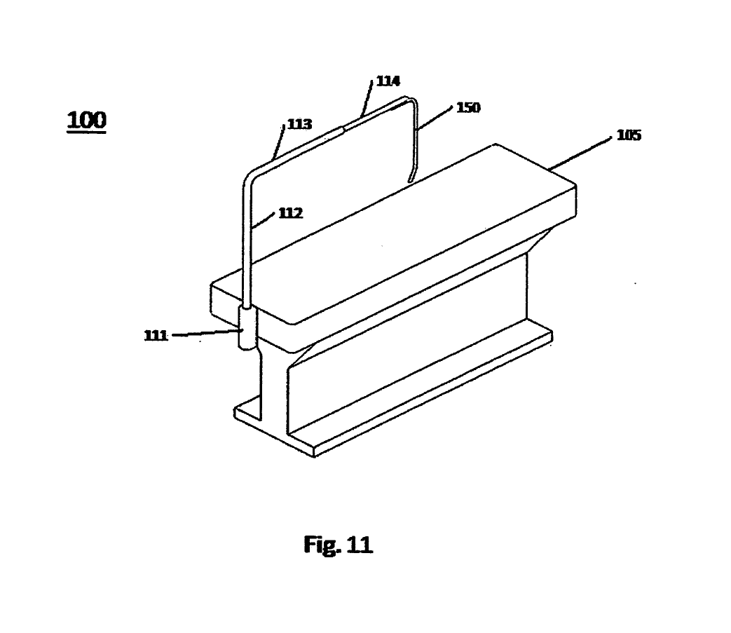

Referring to FIG. 11, a surgical equipment supporting frame 100 is shown. The frame is mounted to the operating table 105 at a head or foot portion thereof in mount 111. The mount 111 may also be located on a left or right side of the operating table 105. The mount 111 is attached to the operating table 105 using for example, a fixing device, such as a clamp, screw, bolt or the like, and includes an opening into which a branch 112 of the frame 100 is inserted. The mount 111 may also be molded or adhered to the operating table 105. The mount 111 snugly holds the branch 112 therein, and may also permit the branch to rotate around the vertical axis, so that the frame 100 can be moved from side to side. The frame 100 also includes an arm portion including branches 113 and 114 substantially perpendicular to branch 112. Like the frame 10, the branches of frame 100 can be molded as a single piece, and/or formed in separate parts and subsequently joined. The branch 114 telescopes into and out of branch 113 to increase or decrease the size of the horizontal portion of the frame 100, so as to extend a surgical tool 150 to different portions of the body of a patient on the operating table 105. One or more surgical tools 150 as described above may be attached to the frame 100 using any of the interfaces, kits and methods for tube or wire dressing described above in connection with frame 10.

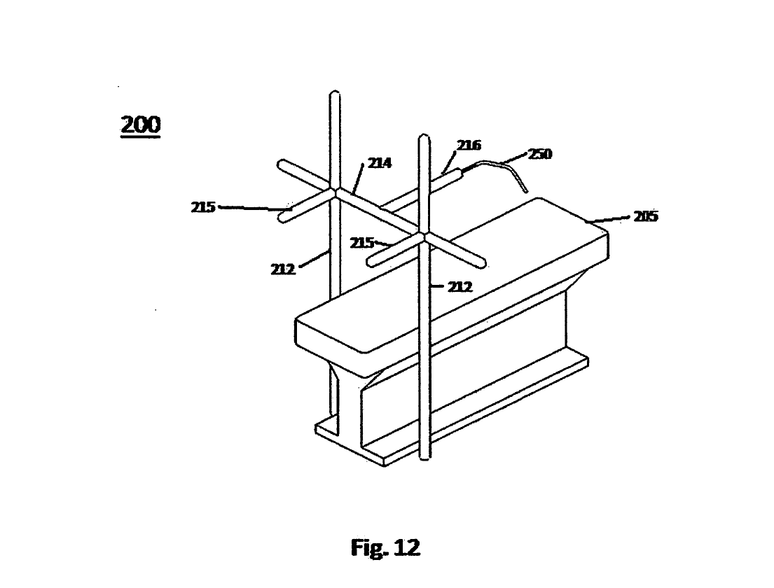

Referring to FIG. 12, a surgical equipment supporting frame 200 is shown. The frame 200 is free standing on the floor of the operating room, and may be moved to different points along a length of the operating table 205 depending on the part of the body that is the focus of the operation. The frame 200 may be braced to the operating table, using for example, a clamp, at a point where branches 212 are in contact with or close to the operating table 205. The frame 200 includes branches 212, 214, 215, and 2l6. Like the frame 10, the branches of frame 200 can be molded as a single piece, and/or formed in separate parts and subsequently joined. One or more surgical tools 250 as described above may be attached to the frame 200 at an arm portion including branch 216 using any of the interfaces, kits and methods for tube or wire dressing described above in connection with frame 10.

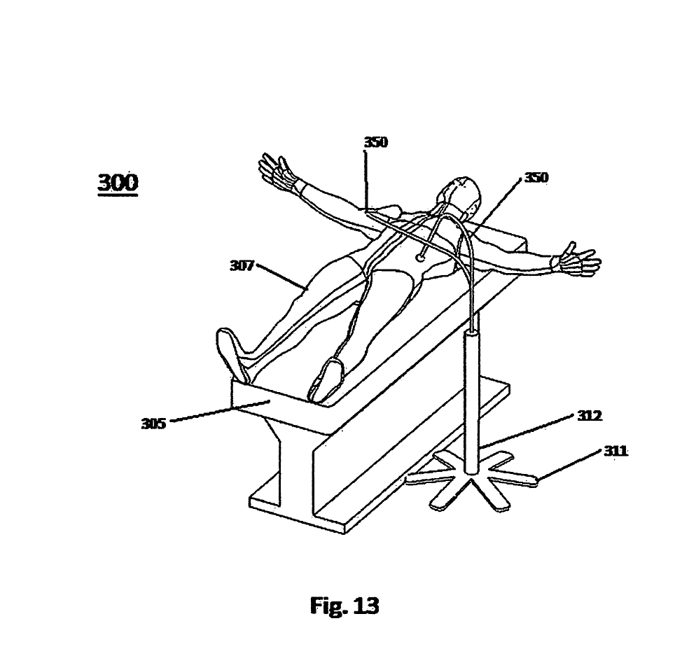

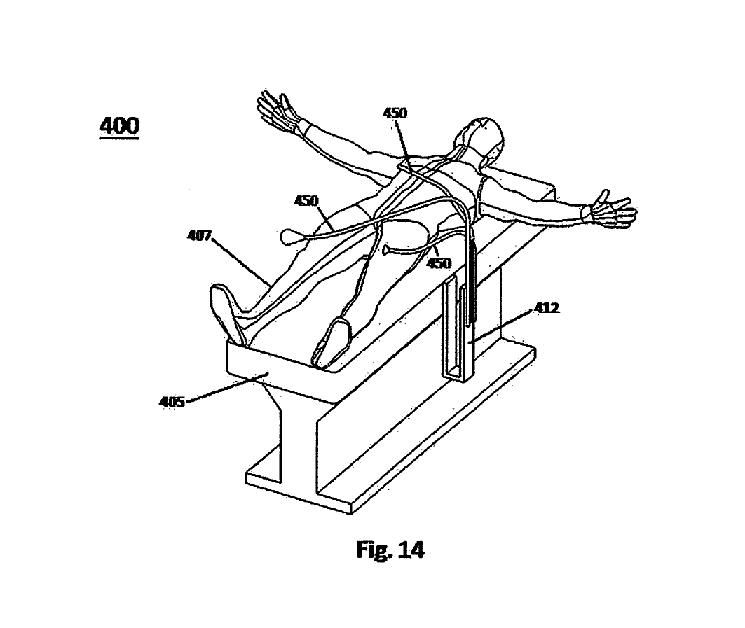

FIGS. 13 and 14 show surgical equipment supporting frames 300 and 400 positioned at a side of an operating table 305, 405.

The frame 300 is free standing on a floor of the operating room, and, like the frame 200, and may be moved to different points along a length of the operating table 305 depending on the part of the body that is the focus of the operation. Further, if necessary, the frame may also be moved to a head or foot of the operating table as needed. The frame 300 includes a base 311, and a vertical branch 312 to which surgical tools 350 as described above are attached. Alternatively, the branch 312 may be attached to a side of the operating table, or to a wall, and the base 311 can be omitted.

The frame 400 includes a U-shaped member 412 attached to a side of the operating table 405 using, for example a clamp or fixing devices, such as screws or bolts. Alternatively, the U-shaped member 412 may be attached to a wall. The U-shaped member 412 may also be molded or adhered to the operating table 405. The U-shaped member 412 may be positioned at different points along a length of the operating table 405 depending on the part of the body that is the focus of the operation. Further, if necessary, the member 412 may also be moved to a head or foot of the operating table as needed. Surgical tools 450 as described above are attached to the U-shaped member 412.

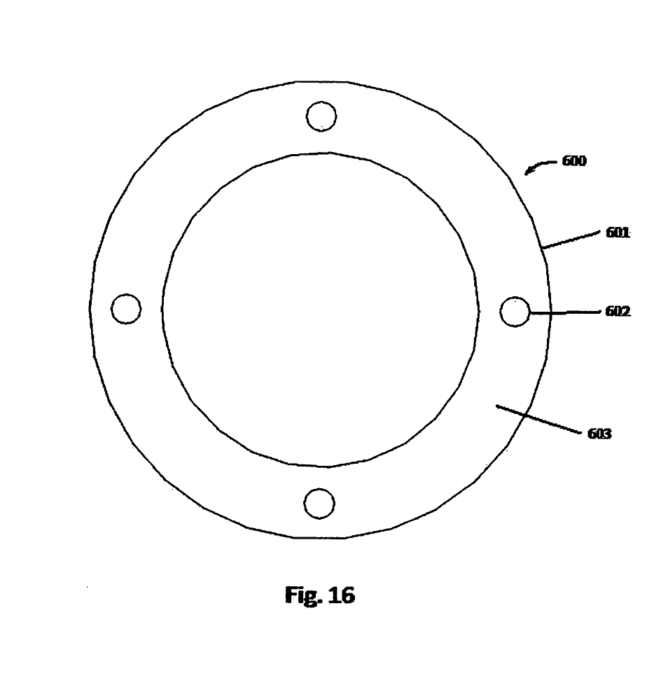

One or more of the surgical tools 350, 450 may be attached to the frame 300, 400 using any of the interfaces, kits and methods for tube or wire dressing described above in connection with frame 10. Since the surgical tools 350, 450 are not suspended from a point above the patient 307, 407, the surgical tools 350, 450 can be used in conjunction with self-supporting tubes as described below in connection with FIGS. 16-20. The self-supporting tubes have the necessary rigidity to support themselves over the body without the need of an overhead frame suspending the tubes therefrom.

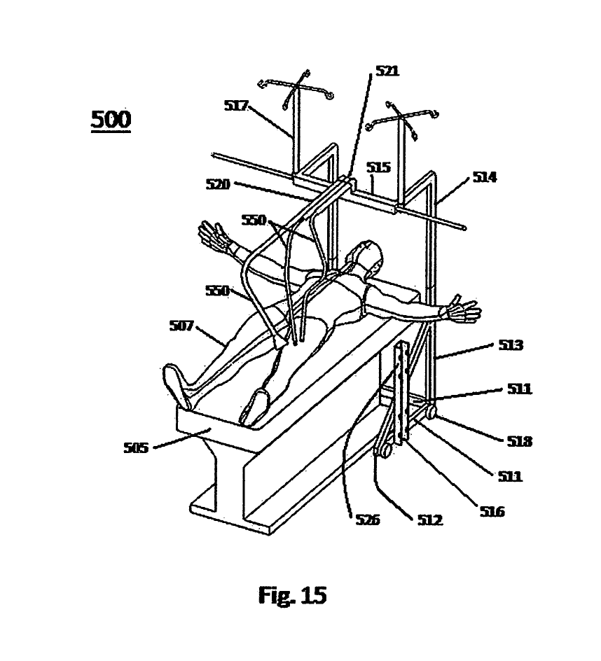

Referring to FIG. 15, a surgical equipment supporting frame 500 is shown. The frame 500 is positioned over and to the side of a patient 507 and the operating table 505. The frame 500 includes branches 511, 512, 513, 514 and 515. Like the frame 10, the branches of frame 500 can be molded as a single piece, and/or formed in separate parts and subsequently joined. In addition, the L-shaped branch 514 and branch 516 are sufficiently high above the patient 507 so as to provide an operator with required clearance for unobstructed access to the body. The branches 512, 512 can be located on both left and right sides of the operating table 505.

One or more surgical tools 550 as described above may be attached to the frame 500 using any of the interfaces, kits and methods for tube or wire dressing described above in connection with frame 10. Further, the tools 550 are attached to the frame 500 via an arm portion including telescoping supports 520. The telescoping supports 520 telescope into and out of base 521 to increase or decrease the size of the telescoping supports 520, so as to extend the surgical tools 550 to different portions of the body of a patient 507 on the operating table 505. The frame 500 includes IV bag supports 517, which may be used support IV bags during an operation. The frame 500 also includes casters or wheels 518 so that the frame 500 may be easily moved into position for an operation, and out of position when the operation is finished. The frame 500 also includes conduit channels 516 through which wires or tubes may pass to their source or deposit points. The conduit channels 516 include holes 526 on sides thereof, which may serve as mounting locations for devices, such as, for example, patient monitors. As is the case with each of the frames 10 and 100-400, the frame 500 may be used in conjunction with self supporting tubes as described below in connection with FIGS. 16-20, or tubes which are not self-supporting.

In some embodiments, the frames 10 and 100-500, in addition to supporting surgical tools, may also support IV bags, sterile curtains or various patient monitors.

The frames 10 and 100-500 are made from, for example, plastic, PVC or metal and can be autoclavable. The arm portions of the frames 10, 100, 200 and 500 are positioned to extend substantially over and/or parallel to a longitudinal (lengthwise) centerline of the operating table to permit delivery of the surgical tools to and along, a centerline of the operating table.

Referring to FIGS. 16-20, self-supporting tubes 600, 700 and 800 are shown. The self-supporting tubes 600, 700, and 800 have the necessary rigidity to support themselves over the body, or within reach of the surgeon, without the need of a frame suspending the tubes therefrom. For example, the self-supporting tubes 600, 700 and 800 are cantilevered from a point of origin, and have enough rigidity so that the cantilevered self-supporting tubes are taut and do not sag.

FIG. 16 is a cross-sectional view of co-extruded tubing 600. The co-extruded tubing includes, for example, PVC tubing 601 with, for example, about 0.4″ OD and about 0.3″ ID. Wires 602 made from, for example, metal or other rigid or semi-rigid material, are co-extruded into the wall 603 of the tube 600 so that the tube can be self supporting and used for functions, such as suction and irrigation. The co-extruded tube 600 can be used in place of a PVC tube that is not co-extruded and not self supporting. There could be one or more support wires 602 that are co-extruded. The cross section of the wire could be various shapes, including for example, circular, square, or oval.

Versions of the tube 600, for example, up to about 3″ in diameter or more, can be used as smoke ducts to convey cautery smoke, laser smoke or other kinds of undesirable airborne particles away from the operating table. The co-extruded tube 600 can also be used to support lights and other tools and utilities.

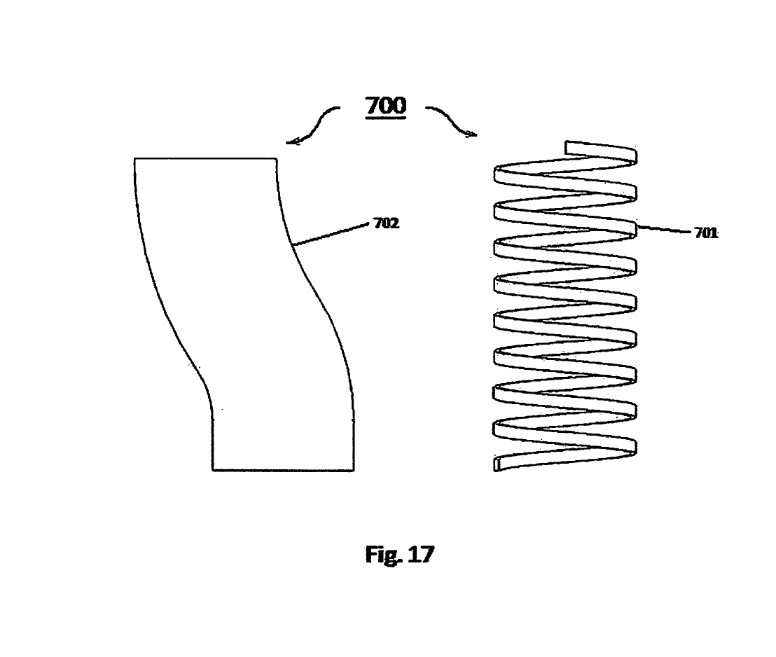

FIG. 17 shows a helix tube 700 in snapped and unsnapped configurations. The helix tube 700 is flexible and hollow, and includes a snap together helix 701, which when snapped together, forms a continuous flexible tube 702 that may range in diameter from, for example, about 0.5″ to about 3″. The material of the helix tube 700 can be, for example, metal or plastic, and is rigid or semi-rigid so that the tube can be self supporting.

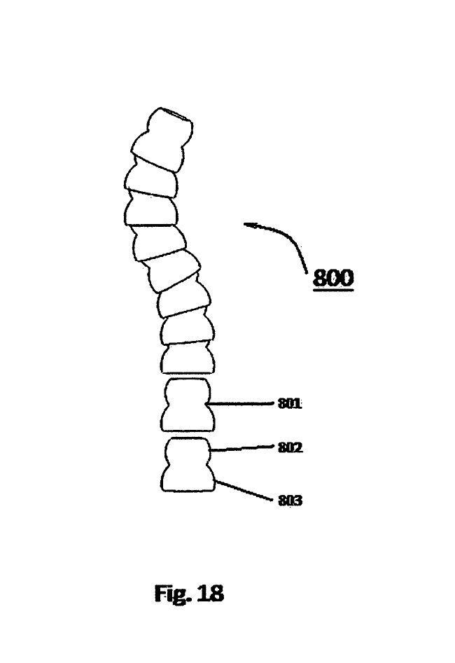

FIG. 18 shows a tube 800 including interlocking knuckles 801 having axial flexibility that permits a self supporting flexible characteristic. Each interlocking knuckle 801 includes top and bottom portions 802 and 803, which fit into respective bottom and top portions of adjacent interlocking knuckles 801. The size of the resulting tube 800 can be in a range of, for example, about 0.5″ to bout 3″ in diameter. The tube material can be, for example, plastic or metal, and the tube 800 is rigid or semi-rigid so that the tube can be self-supporting.

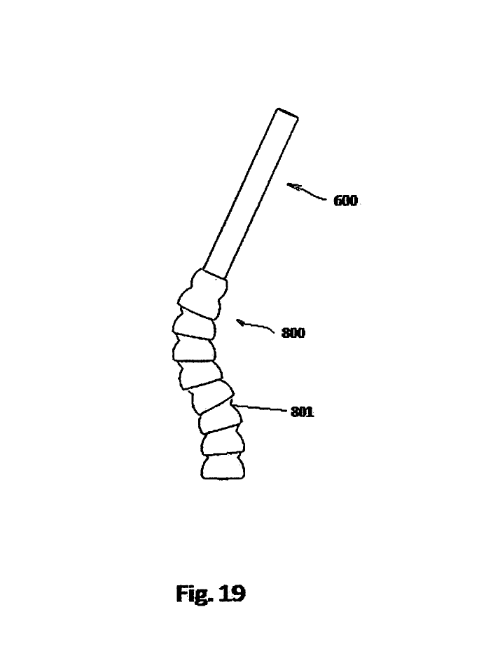

FIG. 19 shows a tube including an interlocking knuckle tube 800 and a co-extruded tube 600 at an end thereof. The tube shown in FIG. 19 is a staged design including a combination of the interlocking and co-extruded designs. Other combinations are also contemplated. For example, it is possible to have a co-extruded tube 600 at the end of a flexible hollow helix tube 700. A combination may also include a co-extruded tube 600 with weaker support wires 602 at the end of a co-extruded tube 600 having stronger wires 602, so as to enable greater flexibility of a tube at the end of the tube. Any combination of the above supports is envisioned and the staged design could consist of more than two stages. At the joints that transition from one stage to another, the tubes, for example tubes 600 and 800 shown in FIG. 19, can be fused together in numerous ways, such as heat welding, ultrasonic welding or with adhesives.

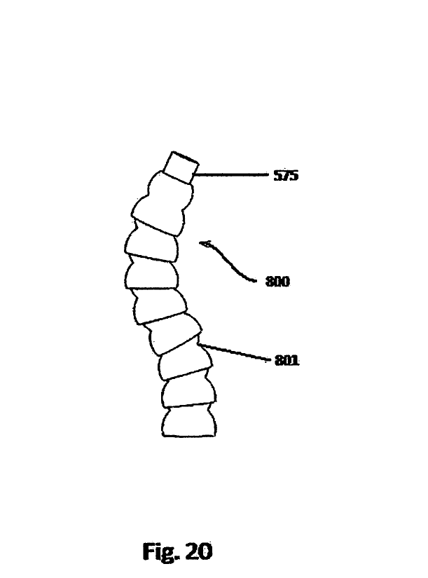

FIG. 20 shows an interlocking knuckle tube 800 including a standard tube 575 therein. For example, standard tubes and/or wires, such as, for example, PVC tubing used for suction or irrigation, can be placed within one of the self supporting structures 600-800 described and shown above. This would enable standard, inexpensive tubes and wires to be used, while providing, for example, the self-supporting features. Alternatively, tubes and wires can also run on the outside of the self-supporting structures 600-800 with clips or other supports holding the tubes and wires to the self supporting structure 600-800.

Each of the tubes 600-800, when used for passing materials directly therethrough, are designed to be leak resistant. In order prevent leakage, tubes 700 and 800, for example, may include a leak proof coating, such as, for example, silicone material, or the like, applied to the tubes 700 and 800.

Another type of self-supporting structure includes a support wire with end clamp connectors. The support wire could be flexible, but also include sufficient rigidity to be self-supporting. The support wire structure utilizes unsupported tubes, wires, smoke ducts and other tools and utilities that clamp to the end of a support wire. The support wire furnishes the self-supporting function for the tubes, wires, etc. attached at an end thereof, and can be, for example, an annealed metal wire with a diameter between, for example, about 0.1″ to about 0.5″.

In addition to tubes to convey fluids and air, such as smoke, and wires to power devices such as a cautery pen, fiber optic cables could also run through the self-supporting structures.

In an alternative embodiment, a supported holder could be mounted to any of the frames 10 and 100-500, and/or attached at an end of any of the self-supporting structures, for example, tubes 600-800, described above. The supported holder includes a tray, a hook, a holster or another holder that allows tools that require very sensitive positioning to be unsecured, yet remain in easy reach for the surgeon. Tethered tools, such as laser cutting hand tools and cautery pens, are examples of tools that may require very sensitive positioning. Other non-tethered tools, including simple tools like scalpels, could also be positioned on the supported holder for easy access.

According to an embodiment, a self supporting smoke exhaust duct formed of the co-extruded tube 600 could have a supported holder mounted to it to support a cautery pen or a laser hand tool. Other self supporting structures, for example tubes 700-800, could also include supported holders mounted thereto.

According to an embodiment, a coupling that allows very controlled motions or high flex life performance could be added to a tube, such as a non-supported tube, anywhere along the tube. This coupling could be a “snap-in” tube holder that snaps onto a tube, receives the tube therethrough, and has a mechanical joint. The coupling could also be an in-line tube of a different construction and/or material that allows improved bending performance. In an embodiment, the in-line tube could be a mostly metal constructed tube that is similar to goose neck type tubing.

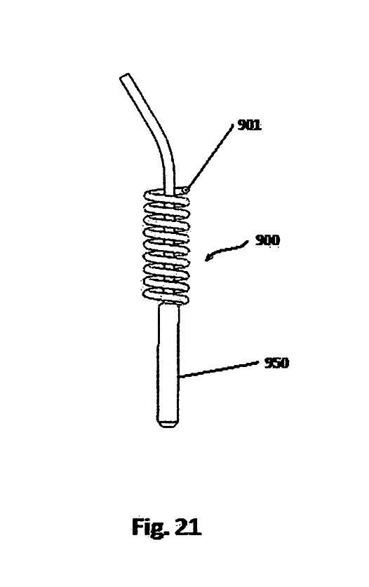

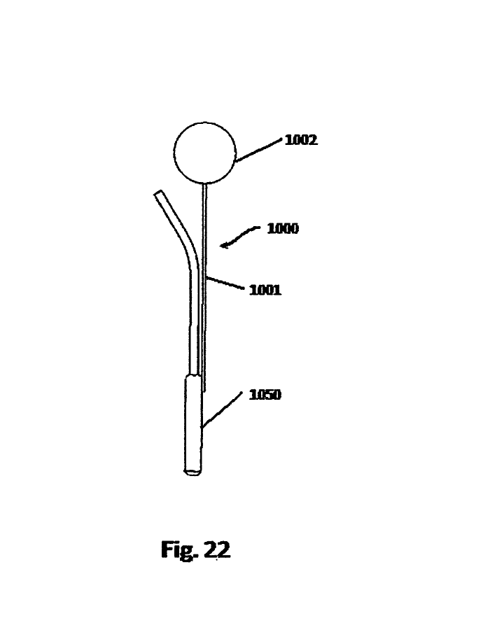

Referring to FIGS. 21 and 22, surgical tools 950, 1050 attached to a pig tail wire support 900 and a recoiling spool support 1000 are shown. These designs allow tools, such as a cautery pen, to hang in place in easy reach of the surgeon. The surgeon can extend the tool support 900, 1000 by simply extending the tool to the desired position. Upon completion of a task, the tool support 900, 1000 retracts and the tool returns to the initial position. The pig tail wire support 900 includes, for example, spiraled wire 901 attached to the tool 950, which can be extended by a pulling force, and returns to its original position when the pulling force is decreased or ceases. The recoiling spool support 1000 is attached to a tool 1050, and includes, for example, wire 1001 which winds around a spring loaded rotating spool 1002. Upon application of a pulling force, the wire 1001 is unwound and can be extended from the spool 1002, and upon completion of a task, the pulling force is decreased to allow spring force to rotate the spool 1002 and retract the wire 1001 around the spool 1002. The spool support 1000 may lock at different positions corresponding to different lengths of the wire 1001. The pig tail wire and recoiling spool supports may be attached to a frame 10 or 100-500, any one of the self-supporting structures 600-800, or attached to a wall, ceiling, or other structure in an operating room. In an embodiment where the pig tail wire and recoiling spool supports are attached to a self-supporting structure, such as structures 600-800, the self-supporting structure will have sufficient rigidity to maintain a substantially stationary position when a pulling force is applied to the pig tail wire and/or recoiling spool support.

In an alternative embodiment, robotic positioning, controlled by, for example, retinal positioning and a confirmation/initiate button controlled by the eye, tongue, hand or foot, can be used by the surgeon to position various devices.

Although exemplary embodiments of the present invention have been described hereinabove, it should be understood that the present invention is not limited to these embodiments, but may be modified by those skilled in the art without departing from the spirit and scope of the present invention, as defined in the appended claims.

What is claimed is:

1. A surgical utility tube, comprising:
an outer surface;
an inner surface;
a wall formed between the inner and outer surfaces; and
a plurality of wires formed in the wall between the outer and inner surfaces.

2. The surgical utility tube of claim 1, wherein the wires are spaced apart at predetermined intervals around the wall of the tube.

3. The surgical utility tube of claim 1, wherein the tube is used for at least one of suction and irrigation.

4. The surgical utility tube of claim 1, wherein the tube is used as a smoke duct.

5. The surgical utility tube of claim 1, wherein the tube is used to support a light.

6. The surgical utility tube of claim 1, wherein the wires at a first end of the tube have less rigidity than the wires at a second end of the tube.

7. The surgical utility tube of claim 1, wherein the wires at an end of the tube have less rigidity than the wires at a portion of the tube between ends of the tube.

8. The surgical utility tube of claim 1, wherein a first end of the tube is attached to a frame positioned at a side of an operating table and a second free end of the tube is self-suspended over the operating table.

9. The surgical utility tube of claim 1, further comprising a pig tail wire attached to the tube, and a surgical tool attached to the pig tail wire.

10. The surgical utility tube of claim 1, further comprising a recoiling spool attached to the tube, and a surgical tool attached to the recoiling spool.

11. A surgical utility tube, comprising:
a plurality of members, wherein the plurality of members are joined together to form a continuous first tube, wherein the first tube is bendable to a plurality of angles along a length of the first tube, and wherein the first tube locks at each of the plurality of angles; and
a second tube positioned in the first tube.

12. The surgical utility tube of claim 11, wherein the second tube is flexible and is used for at least one of suction and irrigation.

13. The surgical utility tube of claim 11, wherein the second tube is flexible and is used as a smoke duct.

14. The surgical utility tube of claim 11, wherein the plurality of members comprise a plurality of interlocking knuckles.

15. The surgical utility tube of claim 11, wherein the plurality of members comprise adjacent sections of a helix.

16. The surgical utility tube of claim 11, wherein a first end of the first tube is attached to a frame positioned at a side of an operating table and a second end of the first tube is self-suspended over the operating table.

17. The surgical utility tube of claim 11, further comprising a pig tail wire attached to the first tube, and a surgical tool attached to the pig tail wire.

18. The surgical utility tube of claim 11, further comprising a recoiling spool attached to the first tube, and a surgical tool attached to the recoiling spool.
